(19) United States
(12) Patent Application Publication
(10) Pub. No.: US 2004/0102358 A1
(43) Pub. Date: May 27, 2004

(54) COMPOSITION FOR TREATING SKIN CONDITIONS

(76) Inventor: ROSEMARIE SCIVOLETTO, DELRAY BEACH, FL (US)

Correspondence Address: OLTMAN FLYNN & KUBLER, 915 MIDDLE RIVER DRIVE, 415 GALLERIA PROFESSIONAL BUILDING, FORT LAUDERDALE, FL, 333043585

(21) Appl. No.: 09/082,292

(22) Filed: May 19, 1998

Related U.S. Application Data
(60) Provisional application No. 60/047,032, filed on May 19, 1997.

(51) Int. Cl. A61K 31/44

(52) U.S. Cl. 514/1; 514/357

(57) ABSTRACT

Compositions for skin treatment are disclosed and include nicotinamide, nicotinic acid, and nicotinic esters as active ingredients. The compositions are applied topically to the skin to treat skin conditions including acne, fine lines and age spots, itching and pain from insect bites, bee stings, fungi (including athletes foot and jock itch), flaking and/or scaly skin (including dandruff, sebouheir dermatitis, psoriasis and heat rash), and burns. Different compositions are presented for use as an acne treatment, a face and body wash, a dermatophyte (nail fungus) treatment, still another is intended for use in makeup, and another in lipstick.

This application is based on provisional application serial No. 60/047032 filed May 19, 1997

### BACKGROUND OF THE INVENTION

1. Field of the Invention

The present invention relates generally to compositions for treating various skin conditions and, more particularly to a topically applied skin treatment composition including a nicotinic ester as an active ingredient.

2. Brief Description of the Prior Art

Various compositions containing nicotinic esters have been known and commonly used for inducing and stimulating hair growth. Examples of these can be found in the Grollier U.S. Pat. Nos. 5,157,036 and 4,968,685. In U.S. Pat. No. 5,468,492, Szaloki discloses a use of vitamin E nicotinate as a rubefacient to improve the circulation of the blood of the scalp. Other compositions containing methyl nicotinate have been proposed as an analgesic for the symptomatic relief of localized pain of musculo-skeletal etiology, as evidenced by the Fisher U.S. Pat. No. 3,880,996.

A product is available and sold under the trade name FINALGON, which is a topical rubefacient including nicotinic acid. The product is intended for use as a temporary relief from pain caused by muscular rheumatism and does not disclose use as a skin treatment.

A variety of published articles describe the effects and uses of nicotinic acid, nicotinamide and methyl nicotinate (Murrell, T., “The Cutaneous Reaction to Nicotinic Acid,” A. M. A. Archives of Dermatology, 79:545-552, May, 1959, Shalita, A. R., “Topical nicotinamide compared with clindamycin gel in the treatment of inflammatory acne vulgaris,” International Journal of Dermatology, 34(6):434-7, June, 1995, and Remme, J. J., “Bullous pemphigold successfully controlled by tetracycline and nicotinamide,” British Journal of Dermatology, 133(1):88-90, July, 1995). None of these articles describe a use of the particular compositions of nicotinamide, its derivatives, nicotinic acid, its esters, or their derivatives as presented by this invention.

In U.S. Pat. Nos. 3,729,685 and 3,906,108 Kligman and Felty, respectively, describe a method of acne treatment which involves topical application of vitamin A to the skin. This method does not, however, include the use of vitamin B3, nicotinic acid or any salt thereof for acne treatment. One disadvantage of using vitamin A in skin treatment is the substantial skin irritation vitamin A causes. This irritation makes such treatment unpleasant and may even lead to an individual foregoing treatment altogether. Although a cream formulation of vitamin A may reduce the undesirable side effects, it does not do so entirely. This leaves an individual who applies the vitamin A treatment with irritated, stinging and itching skin.

The prior art does not provide for the topical application of nicotinamide, its derivatives, nicotinic acid, and nicotinic esters or their derivatives in the compositions presented herein. The present invention presents a spectrum of unique and novel formulas by which nicotinamides, nicotinic acids and nicotinic esters may be topically applied to the skin. Some of the formulas embody an original compilation of ingredients which include skin moisturizers, emollients, vitamin E, carriers and other beneficial elements. Some formulas are designed to dry quickly and clearly upon application. These formulas provide the user with a smooth and even skin tone without the greasy, sticky finish or irritation caused by many other skin care products. Further, these formulas are effective in treating a variety of disorders and skin conditions, including the removal of blackheads.

### SUMMARY OF THE INVENTION

The present invention relates to a discovery that a nicotinic ester and particularly the compound methyl nicotinate, is highly beneficial when topically applied to treat skin conditions. More particularly, when combined with a skin moisturizer, a suitable carrier, an emollient (e.g. glycerol or glycerin), vitamin E and other elements and excipients, methyl nicotinate has surprising efficacy in treating various skin conditions including: acne blemishes; breakout from acne; fine lines and age spots; stretch marks; cellulite; itching; pain and itching from insect bites and stings; fungi; varicose veins; flaking and scaly skin; and burns (including sunburn).

In addition to those listed heretofore, minor amounts of other additives may optionally be present. This would include an ingredient effective in treating dermatophyte (nail fungus).

The invention also encompasses a method of using the composition to treat various skin conditions or other disorders. This method generally includes patting the composition on one's palm or other applicator, such as a cosmetic pad or cotton. The composition is then topically applied to the skin or other affected area such as a fingernail or toenail. The method may also include, in the case of using the composition as a body or facial wash, applying the composition to one's skin then rinsing it away with water.

### DESCRIPTION OF THE PREFERRED EMBODIMENTS

The disclosed embodiments of the present invention display certain preferred compositions but are not intended to limit the scope of the invention. As will be obvious to those skilled in the art, multiple variations and modifications may be made without departing from the scope of the invention.

In the following preferred embodiments, the active ingredient listed is methyl nicotinate. The composition might otherwise utilize nicotinamides, derivatives thereof, nicotinic acids, nicotinic esters or derivatives thereof as an active ingredient.

In a preferred embodiment intended to treat acne, the composition has the following ingredients:

| | |
|---|---|
| Methyl nicotinate | 0.01 to 90% |
| Benzoil peroxide | 0.01 to 20% |
| Aloe vera gel | 35 to 45% |
| Glycerin | 0.8 to 1.8% |
| DMDM hydantoin | 0.02 to 0.25% |
| Tetrasodium EDTA | 0.05 to 0.15% |
| Vitamin E | 0.01 to 0.1% |
| Polysorbate-20 | 0.5 to 1.0% |
| Silk amino acids | 0.01 to 0.1% |
| Hydrolyzed collagen | 0.01 to 0.1% |

and distilled, deionized or demineralized water to bring the total weight concentration to 100%. This composition may also include any combination in whole or in part of resorcinol, sulfur, resorcinol monoacetate, and salicylic acid.

The preferred method of using the composition is to apply it directly to the skin or other affected area. This may be done using the palm of the hands or an applicator such as cotton or a cosmetic pad.

Persons using the composition have reported the clearing up of any blemishes as well as an inhibitory effect wherein new blemishes or breakouts are prevented from forming. The composition's users relate marked improvements in their skin's texture and acne condition. Other users have stated that the product improved their acne as well as the general color and clarity of their skin within a relatively short period of use, two days.

An ingredient or combination thereof intended to treat dermatophyte (nail fungus) may be included in the composition. This ingredient may include 3% chiquinol, 1% haloprogin, 2% miconazole nitrate, 10% providone iodine, 1% tolnafate, or 10 to 25% undecylenic acid or a salt thereof. The undecylenate ingredient may include calcium undecylenate, copper undecylenate, or zinc undecylenate or a combination thereof. The weight concentrations of these ingredients may also vary according to different formulations of the composition.

Some users of the fungus treatment have reported a 95% improvement in their fungus condition. Others have indicated that the composition relieved what had been a disabling finger pain caused by nail fungus to a point of virtual painlessness. Another individual indicated that use of the composition to treat nail fungus has led to increased nail growth and generally harder nails.

In another embodiment of the composition, intended as a general treatment and daily cleanser for face, body and scalp wash and foot bath, the following ingredients are employed:

| | |
|---|---|
| Methyl nicotinate | 0.01 to 20% |
| Aloe vera gel | 60 to 70% |
| Ammonium lauryl sulfate | 20 to 28% |
| Cocamidopropyl betaine | 3 to 5% |
| Lauramide DEA | 3 to 5% |
| Glycerin | 0.8 to 2.5% |
| Methylparaben | 0.8 to 1.2% |
| Propylparaben | 0.8 to 1.2% |
| Propylene glycol | 0.8 to 1.2% |
| Imidazolidinyl urea | 0.8 to 1.2% |
| Guanine | 0.1 to 0.15% |
| Tea-lauryl sulfate | 0.1 to 0.15% |
| Isopropyl alcohol | 0.1 to 0.15% |
| Methylcellulose | 0.1 to 0.15% |
| Vitamin E | 0.01 to 0.1% |
| Silk amino acids | 0.01 to 0.1% |
| Hydrolyzed collagen | 0.01 to 0.1% |
| Chamomile | 0.01 to 0.1% |

and distilled, deionized or demineralized water to bring the total weight concentration to 100%. This composition may also be used on dogs with or without a sulfur additive from 1 to 8%.

This composition would commonly be used by applying the composition to the user's skin, then rinsing it away with water.

This composition is intended for use in treating dry or oily skin, or skin that is a combination of dry and oily, as well as fine lines, enlarged pores, chapped lips, and as an anti-itch formula, on insect stings and bites.

Persons using this composition have reported that it has kept their face from getting oily, prevented breakouts, and reduces fine lines on the face. The composition is said to leave a tingling sensation with increased warmth and has been very useful in treatment of itching sensations caused by rashes and insect bites. The composition is also useful in treating other skin conditions such as psoriasis, eczema, hives or other allergic reactions and associated symptoms that accompany these conditions such as itching.

In addition to treating human skin disorders and or conditions, the various embodiments of the composition presented may be used to treat dogs, cats, horses, and other animals. Two individuals reported use of the composition on a dog having itching problems which had scratched itself to the point of having large irritated bare spots and rashes which sometimes bled. The composition alleviated the dog's discomfort to the point where it no longer scratched and its hair grew back.

Another embodiment of the composition, intended to be applied and left on, includes following ingredients:

| | |
|---|---|
| Methyl nicotinate | 0.01 to 8% |
| Glycerin | 0.8 to 2.5% |
| Isopropyl palmitate | 1 to 15% |
| Myristyl myristate | 1 to 15% |
| Glyceryl ricinoleate | 1 to 15% |
| Octyldodexanol | 1 to 15% |
| Microcrystalline wax | 1 to 10% |
| Acetylated lanolin | 1 to 10% |
| Candelilla wax | 1 to 10% |
| Carnauba | 1 to 10% |
| Isopropyl lanolate | 1 to 15% |
| Cetyl alcohol | 1 to 15% |
| Mineral oil | 1 to 15%. |

This composition may be used in or before applying lipstick.

Another embodiment of the composition is intended for use with makeup and may be used in or before applying makeup. When used before the application of makeup, it is intended to be applied and left on. This embodiment includes the following ingredients:

| | |
|---|---|
| Methyl nicotinate | 0.01 to 8% |
| Glycerin | 0.8 to 2.5% |
| Octylmethothoxycinnamate | 0.8 to 2% |
| Benzophenone-3 | 0.8 to 2% |
| Propylene glycol | 0.8 to 2% |
| Soy lecithin | 0.08 to 2% |
| Glyceryl stearate | 0.08 to 2% |
| Peg-100 stearate | 0.08 to 2% |
| Cetearyl alcohol | 0.1 to .15% |
| Ceteareth-20 | 0.08 to 2% |
| Sodium PCA | 1 to 10% |
| Tocopheryl linoleate | 1 to 10% |
| Tocopheryl acetate | 1 to 10% |
| Methylparaben | 0.8 to 2% |
| Ethylparaben | 0.8 to 2% |
| Propylparaben | 0.8 to 2% |
| DMDM hydantoin | 0.8 to 2% |

This composition would be primarily used in facial makeup along with other dyes, perfumes, colorings, and additives. A similar composition might be used in sun block and sun screen.

While the invention has been described and disclosed in certain embodiments, the scope of the invention is not intended to be, nor should it be deemed to be, limited thereby and such other modifications as may be suggested by the teachings herein are particularly reserved especially as they fall within the breadth and scope of the claims here appended.

I claim:

1. A chemical composition for skin treatment comprising:
an active ingredient selected from the group consisting of nicotinamides, amide derivatives thereof, nicotinic acid, nicotinic esters, and derivatives thereof.

2. The chemical composition of claim 1 wherein the active ingredient is methyl nicotinate.

3. A method of treating skin conditions comprising: topically applying an effective amount of the composition of claim 2 to the skin.

4. A chemical composition for skin treatment comprising:
0.01 to 90% by weight of a compound selected from the group consisting of nicotinamide, amide derivatives thereof, nicotinic acid, nicotinic esters, and derivatives thereof; and,
0.01 to 20% by weight of benzoil peroxide.

5. A method of treating skin conditions comprising: topically applying an effective amount of the composition of claim 4 to the skin.

6. The chemical composition of claim 4 wherein said compound is methyl nicotinate.

7. The chemical composition of claim 6 further comprising: a peeling agent.

8. The chemical composition of claim 7 wherein said peeling agent is a member selected from the group consisting of glycolic acid, benzoil peroxide, and alpha hydroxy.

9. The chemical composition of claim 8 wherein said peeling agent is 0.01 to 90% by weight of the composition.

10. A chemical composition for acne treatment comprising:
by weight, 0.01 to 90% of a compound selected from the group consisting of nicotinamides, amide derivatives thereof, nicotinic acid, nicotinic esters, and derivatives thereof,
by weight, 35 to 45% aloe vera gel;
by weight, 0.8 to 1.8% glycerin;
by weight, 0.02 to 0.25% DMDM hydantoin;
by weight, 0.05 to 0.15% tetrasodium EDTA;
by weight, 0.01 to 0.1% vitamin E;
by weight, 0.5 to 1.0% polysorbate-20;
by weight, 0.01 to 0.1% silk amino acids;
by weight, 0.01 to 0.1% hydrolyzed collagen; and,
water.

11. The chemical composition of claim 10 wherein said compound is methyl nicotinate.

12. The chemical composition of claim 11 further comprising: 0.01 to 20% benzoil peroxide by weight.

13. A method of treating acne conditions comprising: topically applying an effective amount of the composition of claim 12 to the affected skin.

14. The chemical composition of claim 11 further comprising:
by weight, 2% resorcinol; and,
by weight, 3 to 8% sulfur.

15. The chemical composition of claim 11 further comprising:
by weight, 3% resorcinol monoacetate; and,
by weight, 3 to 8% sulfur.

16. The chemical composition of claim 11 further comprising:
0.5 to 2% salicylic acid by weight.

17. The chemical composition of claim 11 further comprising: 3 to 10% sulfur by weight.

18. The chemical composition of claim 12 further comprising:
by weight, 2% resorcinol; and,
by weight, 3 to 8% sulfur.

19. The chemical composition of claim 12 further comprising:
by weight, 3% resorcinol monoacetate; and,
by weight, 3 to 8% sulfur.

20. The chemical composition of claim 12 further comprising:
0.5 to 2% salicylic acid by weight.

21. The chemical composition of claim 12 further comprising: 3 to 10% sulfur by weight.

22. A chemical composition for use as a body and facial wash comprising:
0.01 to 20% by weight of a compound selected from the group consisting of nicotinamides, amide derivatives thereof, nicotinic acid, nicotinic esters, and derivatives thereof.

23. A method of skin cleansing comprising:
topically applying an effective amount of the composition of claim 22 to the user's skin then rinsing the composition off with water.

24. The chemical composition of claim 22 further comprising:
by weight, 60 to 70% aloe vera gel;
by weight, 20 to 28% ammonium lauryl sulfate;
by weight, 3 to 5% cocamidopropyl betaine;
by weight, 3 to 5% lauramide DEA;
by weight, 0.8 to 2.5% glycerin;
by weight, 0.8 to 1.2% methyl paraben;
by weight, 0.8 to 1.2% propyl paraben;
by weight, 0.8 to 1.2% propylene glycol;
by weight, 0.8 to 1.2% imidazolidinyl urea;
by weight, 0.1 to 0.15% guanine;
by weight, 0.1 to 0.15% tea-lauryl sulfate;
by weight, 0.1 to 0.15% isopropyl alcohol;
by weight, 0.1 to 0.15% methylcellulose;
by weight, 0.01 to 0.1% vitamin E;
by weight, 0.01 to 0.1% silk amino acids;
by weight, 0.01 to 0.1% hydrolyzed collagen;
by weight, 0.01 to 0.1% chamomile; and
water.

25. The chemical composition of claim 22 wherein said compound is methyl nicotinate.

26. The composition of claim 25 further comprising:
1 to 8% sulfur by weight of composition.

27. The chemical composition as described in claim 11 further comprising:
a compound useful as an effective ingredient to treat dermatophyte (nail fungus and athlete's foot).

28. A method of treating dermatophyte (nail fungus) comprising:
topically applying an effective amount of the composition of claim 27 to the affected area.

29. The chemical composition of claim 27 wherein said effective ingredient is 3% chiquinol by weight of composition.

30. The chemical composition of claim 27 wherein said effective ingredient is 1% haloprogin by weight of composition.

31. The chemical composition of claim 27 wherein said effective ingredient is 2% miconazole nitrate by weight of composition.

32. The chemical composition of claim 27 wherein said effective ingredient is 10% providone iodine by weight of composition.

33. The chemical composition of claim 27 wherein said effective ingredient is 1% tolnafate by weight of composition.

34. The chemical composition of claim 27 wherein said effective ingredient is 10 to 25% undecylenic acid or a salt thereof by weight of composition.

35. The chemical composition of claim 34 wherein said effective ingredient includes one or more compounds selected from the group consisting of calcium undecylenate, copper undecylenate, and zinc undecylenate.

36. A chemical composition for use in or before lipstick comprising:
0.01 to 8% by weight of a compound selected from the group consisting of nicotinamide, amide derivatives thereof, nicotinic acid, nicotinic esters, and derivatives thereof.

37. A method of applying lipstick comprising using a lipstick which includes an effective amount of the composition of claim 36.

38. The chemical composition of claim 36 wherein said compound is methyl nicotinate.

39. The chemical composition of claim 38 further comprising:
by weight, 0.8 to 2.5% glycerin;
by weight, 1 to 15% isopropyl palmitate;
by weight, 1 to 15% myristyl myristate;
by weight, 1 to 15% glyceryl ricinoleate;
by weight, 1 to 15% octyldodexanol;
by weight, 1 to 10% microcrystalline wax;
by weight, 1 to 10% acetylated lanolin;
by weight, 1 to 10% candelilla wax;
by weight, 1 to 10% carnauba;
by weight, 1 to 10% isopropyl lanolate;
by weight, 1 to 10% cetyl alcohol; and,
by weight, 1 to 15% mineral oil.

40. A chemical composition for use in or before makeup comprising:
0.01 to 8% by weight of a compound selected from the group consisting of nicotinamides, amide derivatives thereof, nicotinic acid, nicotinic esters, and derivatives thereof.

41. A method of applying makeup comprising using a makeup which includes an effective amount of the composition of claim 40.

42. A method of applying makeup comprising first applying the composition of claim 40, then applying the makeup.

43. The chemical composition of claim 40 wherein said compound is methyl nicotinate.

44. The composition of claim 42 further comprising:
by weight, 0.8 to 2.5% glycerin;
by weight, 0.8 to 2% octylmethoxycinnamate;
by weight, 0.8 to 2% benzophenone-3;
by weight, 0.8 to 2% propylene glycol;
by weight, 0.08 to 2% soy lecithin;
by weight, 0.08 to 2% glyceryl stearate;
by weight, 0.08 to 2% peg-100 stearate;
by weight, 0.1 to 0.15% cetearyl alcohol;
by weight, 0.08 to 2% ceteareth-20;
by weight, 1 to 10% sodium PCA;
by weight, 1 to 10% tocopheryl linoleate;
by weight, 1 to 10% tocopheryl acetate;
by weight, 0.8 to 2% methylparaben;
by weight, 0.8 to 2% ethylparaben;
by weight, 0.8 to 2% propylparaben; and
by weight, 0.8 to 2% DMDM hydantoin.
